SEQUENCE LISTING (1 sequence)

SEQ ID NO: 1 - DNA, 1311 bp, Fulvimarina pelagi
atgacgtcccagttcttcggccgctccgaacccgtcgcctatgccggcgagcaaagccgcgatcccctcgcctttcgctggtacgacaaggatcgcgagatcgccggcaagcgaatggaggaccactgccgcttcgccgtctgctattggcattccttcacttggccgggcggcgatccgttcggcggcgagaccttcaatcggccttggatgcacggcgacgatccaatggctcttaccaagcagaaggccgacgtcgccttcgagatgttccggctcctcgatgtgccgttcttcaccttccacgatgtcgacgtcgcgccggaaggcagttcgctccgcgaattcaacgacaatctgaaggccatcaccgacatcttcgctcagaagatggaaagcgcgaaggtgcggctcctctgggggacggcgaacctcttctcaaaccgccggttcatggcgggtgcggcgaccaatccggacccggacgtcttcgccttctcttgtggacaggtgaaagctgcgctcgacgccacgcaccgccttggcggcgcgaactatgtctgctggggtggacgagagggatacgagacgctgctcaacacagacatgaagcgcgaactcgaccagatgggccgtttctattcgatgctggtcgactacaagcataagatcggcttcgaaggccccattctgattgagccgaagccgaaggagccgaccaagcatcagtacgattttgatgccgccgccgtcttcgccttcctgcagaaatacgatcttctcggcgaagtgaagctcaacatcgagcagaaccatgcgatcctcgcgggccacagcttcgaccacgagatccgctacgcctacgccaacgatctcttcggttcgatcgacgttaatcgcggcgacgatctcctaggctgggacaccgaccagttcgccatgaacccgtcggagatggcgctgatgttccacgagatgctgcagcatggcggcttctcgaccggcggactcaacttcgacgccaagatccgccgccagtcgatcgcacccgacgatctcctcatcgcccatgtcgcctcgatggacgcgtgttcgcgcggcctactcgcggcagatcggatgctgaaggacggcgcgctgaccgaaccgctccagaaccgctatgccggctgggacgcgggcgagggaaaagccatcctcgccggcgaacgttctttcgaggaggtcgcaagccgcgccttggatcttgatccgcagcccgtttcagggcgtcaggagatgctcgaaggcattctcaacaggtatgtctga

(19) United States
(12) Patent Application Publication
(10) Pub. No.: US 2014/0011241 A1
(43) Pub. Date: Jan. 9, 2014

(54) Method enabling isomerization process flows at lower pH, lower temperature, and in the presence of certain inhibiting compounds by using the xylose isomerase enzyme from the microorganism Fulvimarina pelagi

(71) Applicants: Christopher Charles Beatty, Albany, NY (US); Stephen Jairus Potochnik, Corvallis, OR (US); Joshua Brandon Kitner, Corvallis, OR (US)

(72) Inventors: Christopher Charles Beatty, Albany, NY (US); Stephen Jairus Potochnik, Corvallis, OR (US); Joshua Brandon Kitner, Corvallis, OR (US)

(21) Appl. No.: 13/723,956

(22) Filed: Dec. 21, 2012

Related U.S. Application Data
(60) Provisional application No. 61/579,629, filed on Dec. 22, 2011.

(51) Int. Cl. C12P 19/24; C12P 7/14

(52) U.S. Cl. CPC C12P 19/24; C12P 7/14
USPC 435/94; 435/162

(57) ABSTRACT

The present invention enables isomerization process flows at lower pH, lower temperature, and in the presence of certain inhibiting compounds by using the xylose isomerase enzyme from the microorganism Fulvimarina pelagi. The xylose isomerase from this marine bacterium not only is very active at the fermentation pH and temperature, it is also tolerant of xylitol and calcium in the amounts generated in biomass fermentation conditions. For the HFCS application, the xylose isomerase from Fulvimarina pelagi is pH compatible with the process and is tolerant of calcium in the amounts found in the HFCS process. The temperature optimum is similar to commercially available amylase/gluco-amylase products.

### SEQUENCE LISTING OR PROGRAM

Not Applicable

### FEDERALLY SPONSORED RESEARCH

The research described in this application was funded in part by a NSF SBIR Grant #1112582.

### CROSS REFERENCE TO RELATED APPLICATIONS

This application claims priority from U.S. Patent Application Ser. No. 61/579,629, entitled “Method enabling isomerization process flows at lower pH, lower temperature, and in the presence of certain inhibiting compounds by using the xylose isomerase enzyme from the microorganism Fulvimarina pelagi”, filed on 22 Dec. 2011. The benefit under 35 USC §119(e) of the United States provisional application is hereby claimed, and the aforementioned application is hereby incorporated herein by reference.

### TECHNICAL FIELD OF THE INVENTION

This invention relates to processes that isomerize sugars with a unique enzyme. Sugars are often isomerized to improve their properties with respect to fermentation or sweetness. The present invention provides improved processes for the production of ethanol from biomass by isomerizing xylose to xylulose and production of high-fructose corn syrup by isomerizing glucose to fructose.

### BACKGROUND OF THE INVENTION

Isomerization is the molecular process of rearrangement where the reactant and product have the same molecular formula (i.e. nothing is added or taken away), but the arrangement is different leading to different properties. The reactant and product are called isomers. Initially isomerization was carried out by simply putting the sugar in a warm solution that was alkaline in nature (1). While this was partially successful, many undesired side products and degradation reactions also occurred. Later it was discovered that that the biological catalysts of living organisms, especially microorganisms, could be harvested and used to isomerize sugars. These catalysts are called enzymes. This innovation describes a process for isomerization that utilizes a novel catalyst from a recently discovered microorganism.

An example of an industrially desirable isomerization is the conversion of xylose to xylulose. Xylose is an abundant sugar in the hemicellulose of biomass, but is not fermentable by conventional yeast such as Saccharomyces cerevisiae or Schizosaccharomyces pombe. This limits the yield of processes that convert biomass into ethanol products such as liquid transportation fuel. Ethanol is desirable fuel because it can be produced from renewable resources and produces less harmful emissions than petroleum based fuels. In order to produce bioethanol economically, it is important to utilize xylose efficiently since it is often the second most abundant sugar in biomass.

It has been previously demonstrated that xylulose is a fermentable sugar (2). This creates an opportunity to devise a high yielding biomass to ethanol process if the appropriate catalyst can be found. (In the classification scheme of enzymes, this group is called xylose isomerase and labeled EC 5.3.1.5.) Several criteria must be met for this to be successful. These include high activity at the fermentation pH, the fermentation temperature, and in the presence of compounds that may inhibit the isomerization reaction. The isomerization and fermentation are preferentially performed simultaneously because of the equilibrium between xylose and xylulose. The reaction equilibrium results in about 20% xylulose and 80% xylose.

Thus, if the isomerization is performed before the fermentation, only about 20% of the xylulose is utilized. If the two processes are occurring simultaneously, the yeast are consuming the xylulose as it is formed and the equilibrium constraint is not reached. Therefore, the preferred embodiment is to perform the isomerization and fermentation simultaneously (Simultaneous Isomerization and Fermentation or SIF). The yeast require that the pH falls between 3.0 and 6.0 for optimal ethanol yield. The fermentation temperature must not exceed about 37° C. for the health of the yeast and is often carried out between 30 and 35° C.(3). Enzyme inhibitors may include competitive inhibitors that act like the substrate (reactant) for the enzyme or metallic elements that interfere with required metallic co-factors for the enzyme. In the case of the xylose to xylulose conversion for fermentation, the primary competitive inhibitor is xylitol. Xylitol is chemically quite similar to xylose and xylulose. Due to this similarity, xylitol often acts as a competitive inhibitor for xylose isomerase.

All of the described xylose isomerases require metallic cofactors for activation of the enzyme (4). These are usually from the group that includes manganese, magnesium, and cobalt. Several metal ions can inhibit these metallo-enzymes. Heavy metals such as lead or mercury are often detrimental to the activity of the enzyme, but they are not typically found in significant quantities in biomass. Calcium is a divalent cation similar to the activating ions and often inhibits xylose isomerase enzymes. Unlike the heavy metals, it is generally found in biomass as it is common in soils and taken up by plants. For instance, a typical wheat straw has about 4000 mg/kg of calcium based on dry weight (5).

More than 60 different microorganisms have been shown to synthesize a xylose isomerase enzyme. Nearly all of these operate from a pH of 7-8 and are incompatible with the fermentation process. There are a few exceptions to this behavior. The xylose isomerase from Lactobacillus brevis is most active at pH 6-7 (6), but is severely inhibited by xylitol (7). Since xylitol is also created by yeast during fermentation, this xylose isomerase has limited utility for this application. The xylose isomerase from Thermus thermophilus and mutants derived from it are also active at pH 6, but the activity at low temperature is very low (U.S. Pat. No. 6,475,768).

Another industrial isomerization process of interest is the conversion of glucose syrup to a mixture of isomers (glucose and fructose) commonly known as High Fructose Corn Syrup (HFCS). In many cases, xylose isomerase enzymes will also isomerize glucose and other sugars. Several of the constraints for this application are similar. The preferred pH is again slightly acidic. This is due to the previous process to make HFCS which hydrolyzes the corn starch into glucose syrup. That process uses an enzyme called alpha-amylase such as that from Aspergillus niger that is used at the slightly acidic pH of 6.0 (8). Alpha amylase requires calcium to function (9). The preferred temperature is substantially higher than fermentation since the previous reaction can be run at up to 70C. The corresponding competitive inhibitor is sorbitol, which may inhibit the xylose isomerase enzyme, but is typically not found in significant quantities in the HFCS process.

### SUMMARY OF THE INVENTION

In contrast to previous work, the present invention enables isomerization process flows at lower pH, lower temperature, and in the presence of certain inhibiting compounds by using the xylose isomerase enzyme from the microorganism Fulvimarina pelagi. The xylose isomerase from this marine bacterium not only is very active at the fermentation pH and temperature, it is also tolerant of xylitol and calcium in the amounts generated in biomass fermentation conditions. For the HFCS application, the xylose isomerase from Fulvimarina pelagi is pH compatible with the process and is tolerant of calcium in the amounts found in the HFCS process. The temperature optimum is similar to commercially available amylase/gluco-amylase products.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

FIG. 1 is a chart of the relative activity of Fulvimarina pelagi xylose isomerase versus pH;

FIG. 2 is a chart illustrating Fulvimarina Xylose Isomerase Activity versus Temperature;

FIG. 3 is a chart illustrating the relative activity of FPXI versus xylitol concentration;

FIG. 4 is a chart illustrating the relative activity for existing commercial and Fulvimarina pelagi XI versus calcium concentration;

FIG. 5 is a chart illustrating the simultaneous Isomerization and Fermentation of xylose with Schizosaccharomyces pombe; and

FIG. 6 is an illustration of a system for providing simultaneous Isomerization and Fermentation System for Existing XI products.

### DETAILED DESCRIPTION OF THE INVENTION

In the following detailed description of the invention and exemplary embodiments of the invention, reference is made to the accompanying drawings (where like numbers represent like elements), which form a part hereof, and in which is shown by way of illustration specific exemplary embodiments in which the invention may be practiced. These embodiments are described in sufficient detail to enable those skilled in the art to practice the invention, but other embodiments may be utilized and logical, mechanical, electrical, and other changes may be made without departing from the scope of the present invention. The following detailed description is therefore, not to be taken in a limiting sense, and the scope of the present invention is defined only by the appended claims.

In the following description, numerous specific details are set forth to provide a thorough understanding of the invention. However, it is understood that the invention may be practiced without these specific details. In other instances, well-known structures and techniques known to one of ordinary skill in the art have not been shown in detail in order not to obscure the invention.

The present invention enables isomerization process flows at lower pH, lower temperature, and in the presence of certain inhibiting compounds by using the xylose isomerase enzyme from the microorganism Fulvimarina pelagi. The xylose isomerase from this marine bacterium not only is very active at the fermentation pH and temperature, it is also tolerant of xylitol and calcium in the amounts generated in biomass fermentation conditions. For the HFCS application, the xylose isomerase from Fulvimarina pelagi is pH compatible with the process and is tolerant of calcium in the amounts found in the HFCS process. The temperature optimum is similar to commercially available amylase/gluco-amylase products.

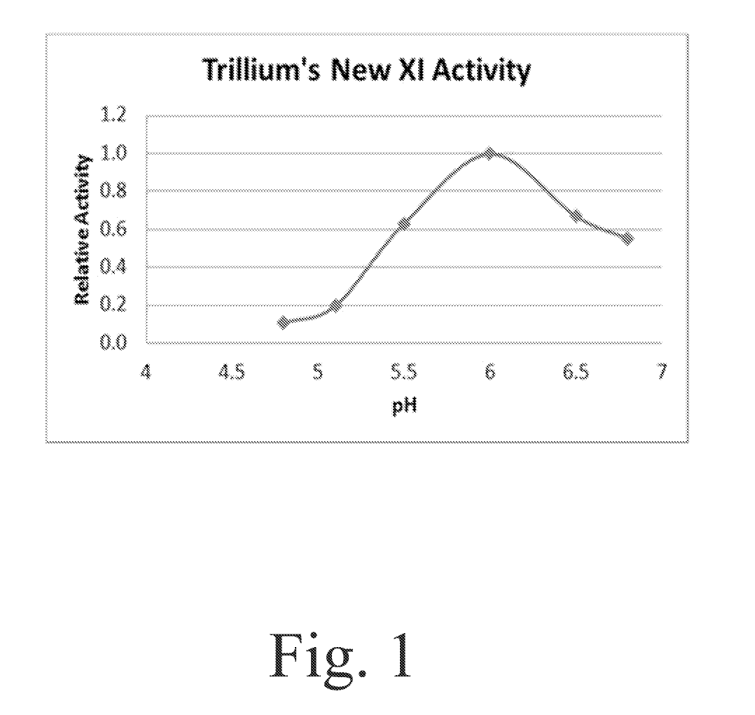

The relative activity of xylose isomerase from Fulvimarina pelagi as a function of pH is shown in FIG. 1. The peak activity at pH 6.0 coincides with the upper bound of optimal pH for yeast fermentation (2). The lower pH is also useful for the high-fructose corn syrup process as it overlaps with the starch saccharification process.

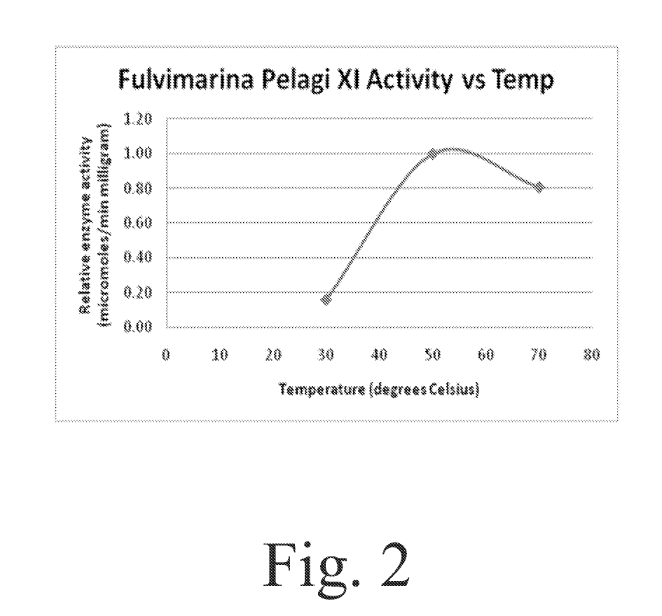

The relative activity of xylose isomerase from Fulvimarina pelagi as a function of temperature is shown in FIG. 2. The enzyme shows high activity at fermentation temperatures of 30-35° C. which is well-suited to SIF processes involving typical yeast. The activity increases up to 50° C. which may be useful for HFCS applications or SIF applications where the enzyme is immobilized and thermally separated from the fermenting yeast or used with a thermophilic bacterium.

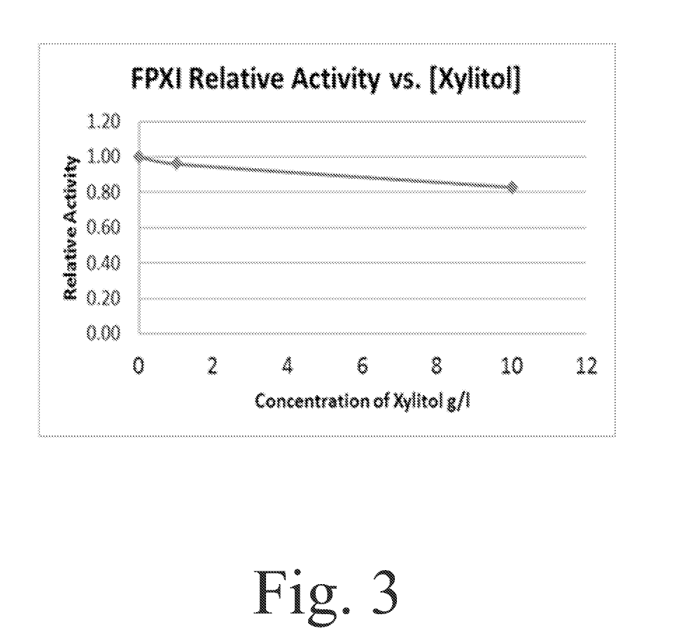

The relative activity of xylose isomerase from Fulvimarina pelagi as a function of xylitol concentration is shown in FIG. 3. Typical concentrations in xylose fermentation are 1-5 g/1 (see data below) which has a negligible effect on the XI from Fulvimarina pelagi. Xylitol is not present in corn starch operations to any significant degree.

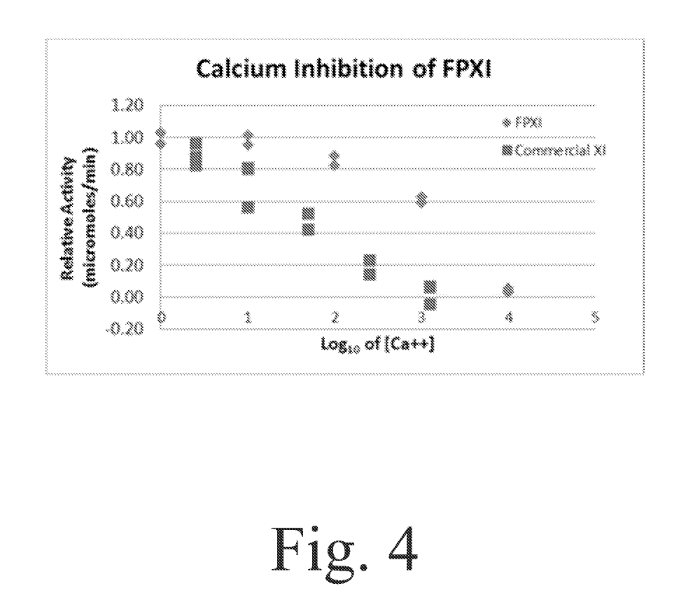

The relative activity of xylose isomerase from Fulvimarina pelagi as a function of calcium concentration is shown in FIG. 4. For comparison, the calcium inhibition curve for a commercially available xylose isomerase (GENSWEET, from GENENCOR) is included. For a biomass that has 0.4% Ca++ and is hydrolyzed in a 10% slurry, this results in 400 ppm of calcium (logᵢₙ 400=2.6 on graph) in the fermentation mixture. At this level of calcium, the commercial XI would retain only 20% of its activity while the Fulvimarina pelagi XI would retain 80% of its activity—a dramatic 4×improvement.

For the SIF process, there are several functional embodiments, depending on the feedstock and other variables. These examples are meant to be representative, but not limit the scope of the invention as other process flows are possible which utilize the innovation.

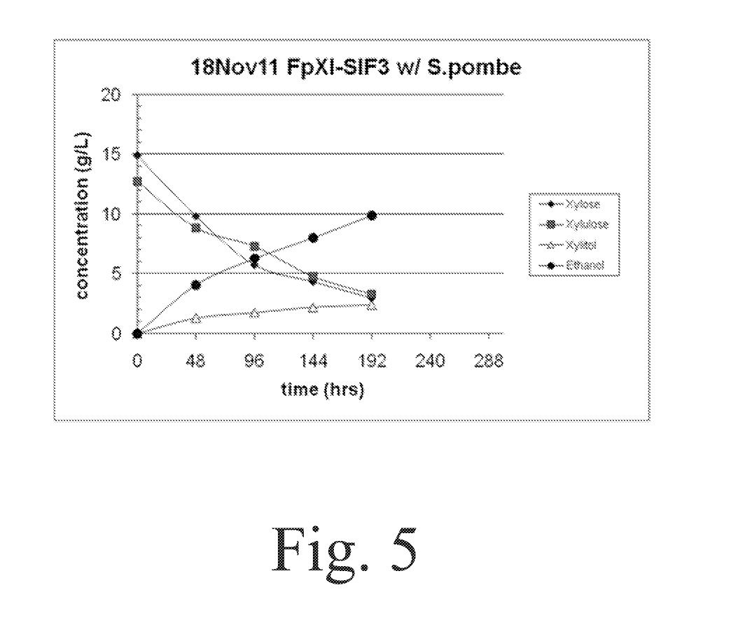

In one embodiment, the xylose isomerase enzyme is extracted from the producing microorganism and used in solution during an SIF process. In this case, Fulvimarina pelagi is grown in a reaction vessel and then the cells are concentrated, typically by centrifugation. The cells are then lysed with sonification, pressure drop methods, lysis reagents, or a combination of these. The material is then centrifuged and the supernatant which contains the xylose isomerase (and other materials) is collected. This material is called “cell-free extract” and is the unpurified form of the enzyme. It may be used in this form or may go through a series of purification steps depending on the application. In some cases, it is desirable to purify by means of precipitation reactions (protamine sulfate, ammonium sulfate), affinity reactions (e.g. magnesium or antibodies), or chromatography. FIG. 5 shows a Simultaneous Isomerization and Fermentation of reagent xylose using the yeast Schizosaccharomyces pombe and the cell free extract of Fulvimarina pelagi. Xylose is being converted to xylulose by the enzyme while the yeast is converting the xylulose to ethanol. The yield of ethanol is greater than 90% of the theoretical yield on fermented sugar.

For the conversion of biomass to ethanol, the SIF (simultaneous isomerization and fermentation) may take place concurrently or separately with the hydrolysis (also called saccharification) of the biomass to component sugars. When performed together, the process is referred to as Simultaneous Saccharification, Isomerization and Fermentation (SSIF). The advantage to this process is the equipment simplicity as several processes occur within a single vessel. Even if the saccharification and SIF are performed separately or in a hybrid fashion (since saccharification often takes place at approximately 50° C.), the same vessel may be used if the temperature of the vessel can be changed. Additional embodiments are described below which may require greater equipment complexity, but may have other advantages such as greater enzyme utilization.

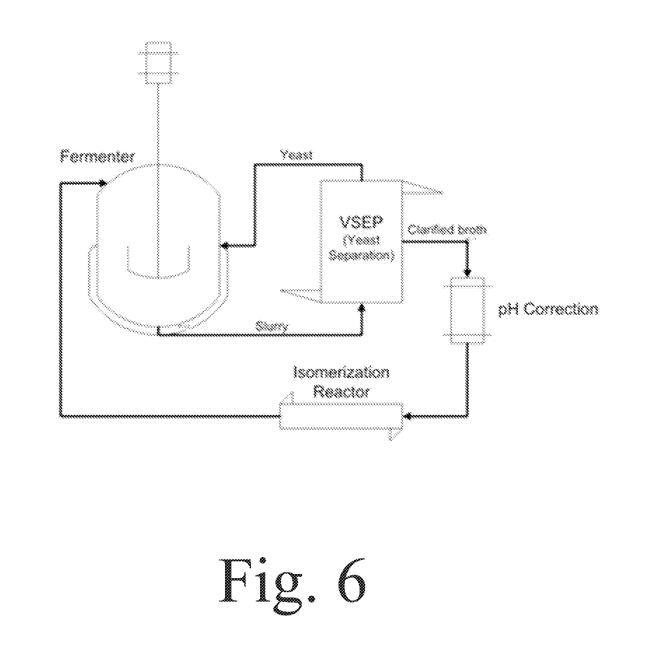

In another embodiment, the xylose isomerase is extracted from the producing microorganism and subsequently immobilized on a carrier. The material to be isomerized, such as fermentation broth or glucose syrup, is passed over the bed or immobilized enzyme. The support may be many different materials included but not limited to polymeric materials, silica fibers, gels, or particles, diatomaceous earth, chitin, or other materials with high surface area and low cost. The material to be isomerized may be passed over the bed a single time with sufficient time to approach equilibrium (as is typically done for glucose syrup) or it may be passed over the bed multiple times as is the case for SIF as the xylose equilibrium is only 20% xylulose product and subsequent passes after fermentation has depleted the fermentable xylulose allow more of the material to be fermented as shown in FIG. 6.

In either of these embodiments, the producing microorganism may be the native Fulvimarina pelagi or from a heterologously expressed gene in a more productive host. The native host produces functional enzyme, but the growth conditions and achievable culture densities are suboptimal compared to what can be achieved with a protein expression system. The enzyme may be used in a purified, partially purified, or unpurified form whether it is harvested from the native host or an expression host. Common microbiological expression systems that are used include E. Coli, Pichia Pastoris, Bacillus subtilus, Schizosaccharomyces pombe, and Saccharomyces cerevisiae. Mammalian, baculovirus, and insect based systems are also possible. The microbiological systems are generally able to produce functional bacterial proteins at the lowest cost.

Expression of FPXI in E. coli using the pET-SUMO system.

For recombinant expression of F. pelagi's xylose isomerase (FpXI), the CHAMPION pET SUMO Protein Expression System from Invitrogen was used. A 6×histidine tag (HIS) is appended to the XI gene to simplify purification on an immobilized metal affinity chromatography (IMAC) column. Competent E. coli cells, BL21(DE3), were transformed with vector pET-SUMO HIS-FpXI by the heat shock method and allowed to recover in Super Optimal broth with Catabolite repression (S.O.C.) media with kanamycin (50 ug/mL) at 37C overnight on an orbital shaker. The kanamycin kills cells that do not have the desired gene. The overnight culture was used to inoculate a flask of Luria-Bertani (LB) media with 1% glucose and kanamycin (50 ug/mL). When the OD600 measurement reaches between 0.400-0.600, the induction of recombinant FpXI expression was initiated with Isopropyl β-D-1-thiogalactopyranoside (IPTG) to a final concentration of 2.4 mg/mL. The incubation temperature was then lowered to 20C and the flask was set on an incubator-shaker for 4 hours. The pH of the culture medium was adjusted to maintain pH 7 using 3 M NaOH drop-wise. Cells were harvested after four hours of induction by centrifugation and washed in 50 mM succinate buffer pH7.0.

Washed cells were lysed in Y-per reagent (Thermo-Fisher-Pierce Scientific) and debris was removed by centrifugation. The rFpXI enzyme was partially purified from the cell free extract using Thermo Scientific's HisPur™ Cobalt Purification Kit. The recombinant XI showed similar activity to the native XI at pH 6.0 and 30C.

The protein sequence for Fulvimarina pelagi's xylose isomerase is the following amino acids as shown in Table 1:

TABLE 1
Protein sequence for Fulvimarina pelagi's xylose isomerase.

| | | | | | | |
|---|---|---|---|---|---|---|
| 1 | mtsqffgrse | pvayageqsr | dplafrwydk | dreiagkrme | dhcrfavcyw | hsftwpggdp |
| 61 | fggetfnrpw | mhgddpmalt | kqkadvafem | frlldvpfft | fhdvdvapeg | sslrefndnl |
| 121 | kaitdifaqk | mesakvrllw | gtanlfsnrr | fmagaatnpd | pdvfafscgq | vkaaldathr |
| 181 | lgganyvcwg | gregyetlln | tdmkreldqm | grfysmlvdy | khkigfegpi | liepkpkept |
| 241 | khqydfdaaa | vfaflqkydl | lgevklnieq | nhailaghsf | dheiryayan | dlfgsidvnr |
| 301 | gddllgwdtd | qfamnpsema | lmfhemlqhg | gfstgglnfd | akirrqsiap | ddlliahvas |
| 361 | mdacsrglla | adrmlkdgal | teplqnryag | wdagegkail | agersfeeva | sraldldpqp |
| 421 | vsgrqemleg | ilnryv | | | | |

In another embodiment, the xylose isomerase gene responsible for the production of the xylose isomerase with desirable properties is heterologously expressed in another microorganism that can perform additional functions that are desired. In the case of SIF, the XI gene may be expressed in a fermentative microorganism so that the yeast or bacterium is able to isomerize the xylose to xylulose and then ferment the xylulose to ethanol. Typical yeast that are used include Saccharomyces cerevisiae, Schizosaccharomyces pombe, Kluveromyces marxianus, and Pichia stipitis.

Thus, it is appreciated that the optimum dimensional relationships for the parts of the invention, to include variation in size, materials, shape, form, function, and manner of operation, assembly and use, are deemed readily apparent and obvious to one of ordinary skill in the art, and all equivalent relationships to those illustrated in the drawings and described in the above description are intended to be encompassed by the present invention.

In addition, other areas of art may benefit from this method and adjustments to the design are anticipated. Thus, the scope of the invention should be determined by the appended claims and their legal equivalents, rather than by the example given.

The embodiments of the invention in which an exclusive property or privilege is claimed are defined as follows:

1. A method enabling isomerization process flows using the xylose isomerase enzyme from the microorganism Fulvimarina pelagi comprising the steps of:
extracting the xylose isomerase enzyme from the producing microorganism Fulvimarina pelagi by:
growing the host microorganism in a reaction vessel and then concentrating the cells, typically by centrifugation;
harvesting the xylose isomerase enzyme by lysing the cells with a combination of sonification, pressure drop methods, lysis reagents and centrifuging the concentrated cells to create a supernatant which contains the xylose isomerase or collecting the supernatant above the concentrated cells which contains a secreted enzyme; and
isomerizing the sugar by placing it in contact with the xylose isomerase enzyme.

2. The method of claim 1 wherein, the xylose isomerase from Fulvimarina pelagi is pH compatible and is tolerant of the calcium amounts.

3. The method of claim 1 wherein, the xylose isomerase from Fulvimarina pelagi is tolerant of xylitol and calcium in the amounts generated in biomass fermentation conditions.

4. The method of claim 1 wherein, the peak activity at pH 6.0 coincides with the upper bound of optimal pH for yeast fermentation.

5. The method of claim 1 wherein, the xylose isomerase enzyme from the microorganism Fulvimarina pelagi shows high activity at fermentation temperatures of 30-35° C. which is well-suited to SIF processes involving typical yeast.

6. The method of claim 1, further comprising the steps of:
providing a fermentation temperature of 30-50° C.; and
immobilizing and thermally separating the xylose isomerase enzyme from the microorganism Fulvimarina pelagi from the fermenting yeast.

7. The method of claim 1, further comprising the steps of:
providing a fermentation temperature of 30-50° C.; and
combing the xylose isomerase enzyme from the microorganism Fulvimarina pelagi with a thermophilic bacterium.

8. The method of claim 1 wherein, the sugar is glucose isomerized to fructose for the production of high-fructose corn syrup.

9. A method enabling isomerization process flows using the xylose isomerase enzyme from the microorganism Fulvimarina pelagi comprising the steps of:
expressing the xylose isomerase gene from Fulvimarina pelagi in a host microorganism;
propagating a culture of the recombinant microorganism; and
isomerizing the sugar by placing it contact with the presence of recombinant microorganism.

10. The method of claim 9 wherein, the recombinant microorganism has the ability, through native or additional expressed genes, to ferment the isomerized sugar to ethanol.

11. A method enabling isomerization process flows using the xylose isomerase enzyme from the microorganism Fulvimarina pelagi comprising the steps of:
extracting the xylose isomerase enzyme from the native or recombinant producing microorganism;
using the extracted the isomerase enzyme in solution during an SIF further process comprising the steps of:
growing the microorganism in a reaction vessel;
concentrating the microorganism cells are concentrated, typically by centrifugation. The
the cells are then lysed; and
the material is then centrifuged and the supernatant which contains the xylose isomerase enzyme is collected.

12. The method of claim 11 wherein, wherein the the microorganism cells are lysed with sonification, pressure drop methods, lysis reagents, or a combination of these.

13. The method of claim 11 wherein, the supernatant is purified by means of precipitation reactions (protamine sulfate, ammonium sulfate), affinity reactions (e.g. magnesium or antibodies), or chromatography.

14. The method of claim 11, further comprising the steps of:
creating a simultaneous isomerization and fermentation of reagent xylose using the yeast Schizosaccharomyces pombe and the cell free extract of Fulvimarina pelagi xylose isomerase;
using the enzyme to convert Xylose to xylulose;
using the yeast to convert the xylulose to ethanol.

15. The method of claim 14, wherein the yield of ethanol is greater than 90% of the theoretical yield on fermented sugar.

16. The method of claim 11, further comprising the steps of:
concurrently performing the hydrolysis of the biomass to component sugars.

17. The method of claim 11, further comprising the steps of:
separately performing the hydrolysis of the biomass to component sugars.

18. The method of claim 17, wherein the same vessel is used if the temperature of the vessel can be changed.

19. A method enabling isomerization process flows using the xylose isomerase enzyme from the microorganism Fulvimarina pelagi comprising the steps of:
the xylose isomerase is extracted from the native or recombinant producing microorganism and subsequently immobilized on a carrier;
the material to be isomerized, such as fermentation broth or glucose syrup, is passed over the bed or immobilized enzyme; and
the material to be isomerized is passed over the bed a single time with sufficient time to approach equilibrium.

20. The method of claim 19, wherein the support may be many different materials included but not limited to polymeric materials, silica fibers, gels, or particles, diatomaceous earth, chitin, or other materials with high surface area and low cost.

21. The method of claim 19, wherein the material to be isomerized is passed over the bed is passed over the bed multiple times as is the case for SIF as the xylose equilibrium is only 20% xylulose product and subsequent passes after fermentation has depleted the fermentable xylulose to allow more of the material to be fermented.
